(19) United States
(12) Patent Application Publication
(10) Pub. No.: US 2007/0270712 A1
(43) Pub. Date: Nov. 22, 2007

(54) Arrangement for Taking a Sample of Bone Marrow and/or Evacuating the Sinuses

(75) Inventors: Hans Wiksell, Taby (SE); Gert Auer, Solna (SE); Vilhelm Ekstrand, Nacka (SE); Eva Munck-Wikland, Stockholm (SE); Peter Harge, Saltsjobaden (SE)

Correspondence Address: YOUNG & THOMPSON, 745 SOUTH 23RD STREET, 2ND FLOOR, ARLINGTON, VA, 22202, US

(73) Assignee: VIBRATECH AB, Stockholm (SE)

(21) Appl. No.: 11/664,349

(22) PCT Filed: Oct. 3, 2005
(86) PCT No.: PCT/SE05/01452

§ 371 Date: Apr. 2, 2007

Related U.S. Application Data
(60) Provisional application No. 60/614,991, filed on Oct. 4, 2004.

(30) Foreign Application Priority Data
Oct. 4, 2004 (SE) 0402394-1

(51) Int. Cl. A61B 10/02

(52) U.S. Cl. 600/567

(57) ABSTRACT

An arrangement for sinus evacuation and for taking a sample of bone marrow during a bone marrow aspiration procedure as well as a bone marrow biopsy procedure. By applying a longitudinal movement to the needle when the needle is penetrating the bone the penetration force is reduced which lower the risk of penetrating the posterior cortex or the second sinus wall. The arrangement further includes a stopping element that prevents the needle to penetrate deeper than to a predetermined depth. The arrangement further includes longitudinal movement a stopping element that provides for an active break of the longitudinal movement of the needle when the needle has penetrated down to the predetermined depth.

### FIELD OF THE INVENTION

The present invention relates to an arrangement for taking a sample of bone marrow and for evacuating the sinuses. Cell sampling is preferably performed during a bone marrow aspiration procedure as well as a bone marrow biopsy procedure.

### BACKGROUND OF THE INVENTION

Peripheral blood examination and other routine laboratory assays do not always provide enough information for the diagnosis of hematologic diseases. In some patients, direct microscopic examination of the bone marrow is required for confirmation of a suspected clinical diagnosis or for monitoring the course of medical therapy.

During the bone marrow examination, bone marrow material is aspirated and spread on slides or a biopsy core of bone and marrow may be obtained. The bone marrow aspirate preparations are valuable for differential cell counts and the evaluation of individual cell morphology, while the biopsy procedure is preferred in for instance the evaluation of marrow cellularity, accurate determination of the number of megakaryocytes and the detection of local lesions such as leucemias and metastatic malignacies. Since the aspirate and biopsy provide complementary information, both specimens are routinely obtained. Ongoing advances in genetics and functional genomics suggest that a completely objective molecular diagnostic procedure in single cells from fine needle aspirates will be available in a near future.

Bone marrow material may for instance be aspirated from the posterior or anterior iliac crest, sternum and tibia, the latter is generally used only with small children. During the aspiration procedure, the aspirate needle (<1.2 mm) is held horizontally or vertically towards the penetration site whereupon the needle generally is advanced with steady pressure and a slight twisting motion through the skin and subcutaneous tissue. The needle is then gently advanced through the cortical bone by rotation and steady forward pressure. The cortical bone may be very soft in for instance elderly patients or very hard in for instance patients with hyperostosis. A sensation of decreased resistance usually indicates penetration of the cortex and entry of the needle into the spongy cancellous bone, which often causes a painful sensation in the patient. The needle is then further advanced about 1 cm into the marrow cavity whereupon a sample of bone marrow cells is aspirated, which for instance may be smeared on a glass slide for examination. If the aspiration procedure is performed in the sternum, care should be taken so that the needle is not penetrating the posterior table of the sternum in order to avoid the risk of injuring underlying vital organs. Marrow aspiration from the sternum is usually performed only when the posterior and anterior iliac crests are diseased or otherwise inaccessible.

During a bone marrow biopsy procedure the coarse needle (1.2-3.2 mm) is inserted through the skin puncture site, advanced with steady pressure to the periosteum and twisted into the surface of the cortex bone. This is usually a painful experience for most patients. Decreased resistance usually indicates entry into the marrow cavity. The biopsy core is broken off from the surrounding bone by rotating the needle 360° several times while applying slight pressure. Decreased resistance to rotation usually indicates detachment of the core from the surrounding bone. The needle is then carefully withdrawn with rotation through the bone, periosteum and skin whereupon the biopsy core is removed from the needle and transferred for further examination.

Due to the larger calibre of the bone marrow biopsy needle, more penetration force is usually required than with the aspirate needle. The applied force to the needle may be up to 300 N in order to penetrate the hard cortex bone. Thus, it is very difficult to stop the motion of the needle when entering the bone marrow cavity and there is a risk of penetrating the posterior cortex, i.e. the second bone wall. Therefore, bone marrow biopsy procedure is generally avoided in the sternum due to the risk of large fatal internal bleeding and the risk of injury to vital organs beneath the sternum as discussed above. Moreover, the use of even coarser needles, i.e. >3.2 mm, is currently not possible due to the high penetration forces needed.

When the sinuses are infected, evacuation of purulent secretion is often desirable and sometimes necessary. During the evacuation procedure, the needle (approximately 1.4 mm) is placed at the penetration site under the inferior concher in the nose whereupon the needle is advanced through the bone by steady forward pressure. A sensation of decreased resistance usually indicates penetration of the medial sinus wall. The needle is then further advanced about 2 mm into the cavity whereupon aspiration of the secretion for bacterial culture is usually performed whereupon the cavity is flushed with saline solution through the inserted needle. The pain and discomfort associated with this procedure is often substantial. As discussed in the previous methods it is often difficult to stop the motion upon penetration of the bone due to the high applied force that is required. Penetration of the second sinus wall may cause serious bleeding and complications in the orbit. In some patients the medial sinus wall is so hard that penetration is impossible with the currently available equipment.

WO 03/101306 A1 (Vidacare Corporation) describes an apparatus for removing a portion of bone marrow. The apparatus is provided with means that applies a rotational movement to the needle when penetrating the bone.

US 2003/0176811 A1 (Shapira) describes an apparatus with a needle for extracting bone material from an extraction site using a vacuum source. The needle is provided with an abrading member that is adapted to break up and liquefy bone material with a rotating or reciprocating motion.

Another issue to attend during the bone marrow biopsy procedure is the prevention of the biopsy core to be left in-situ during specimen extraction, that is to prevent that the biopsy core falls out of the needle “dry tap”. It is therefore known to provide the needle with core-securing means that captures the biopsy core in the needle, see Goldenberg A S, Tiesing a J J. Clinical experience with a new specimen capturing bone marrow biopsy needle. Am J Hematol 2001; 68:189-193.

The inventors of the present invention have thus identified a need of an arrangement that reduces the penetration force needed on the needle in order to penetrate the bone for evacuation of the sinuses and for bone marrow aspiration procedures as well as bone marrow biopsy procedures. There is also a need for an arrangement for taking a sample of bone marrow or evacuating the sinuses that reduces the risk of penetrating the posterior cortex or the second sinus wall.

Moreover, there is a need for an arrangement for taking a sample of bone marrow that reduces the risk of leaving a biopsy core in-situ. Further, there is a need for an arrangement that may be used with biopsy needles with a diameter >3.2 mm, i.e. a diameter larger than conventionally used in the art.

There is also a need for an arrangement that reduces the experienced discomfort in the patient during the insertion of the needle and also reduces the total procedure time.

### SUMMARY OF THE INVENTION

The object of the present invention is thus to provide an arrangement for evacuation of the sinuses and taking a sample of bone marrow during bone marrow aspiration procedures as well as bone marrow biopsy procedures that reduces the penetration force needed on the needle in order to penetrate the bone, particularly in order to reduce discomfort and the risk of penetrating the posterior cortex or the second sinus wall as well as be able to penetrate sites otherwise inaccessible due to thick or hard bone structures. This object is achieved by an arrangement according to the preamble of the independent claim and provided by the features according to the characterizing portion of the independent claim.

Another object of the present invention is to even further reduce the risk of penetrating the posterior cortex or second sinus wall. Still another object of the present invention is to reduce the risk of leaving a biopsy core in-situ.

Preferred embodiments are set forth in the dependent claims.

Due to the lower penetration force needed on the needle, said arrangement may be used with biopsy needles with a diameter >3.2 mm, i.e. a diameter larger than conventionally used in the art. The arrangement also increases the accuracy during the procedure and provides less discomfort and reduces the experienced pain in the patient during the insertion of the needle into the bone marrow or the sinuses. The arrangement also reduces the sampling procedure time as well as enables the operator to penetrate sites otherwise inaccessible due to thick or hard bone masses.

### SHORT DESCRIPTION OF THE DRAWINGS

FIG. 1 shows a schematic side view of the inventive arrangement, partially in cross section.

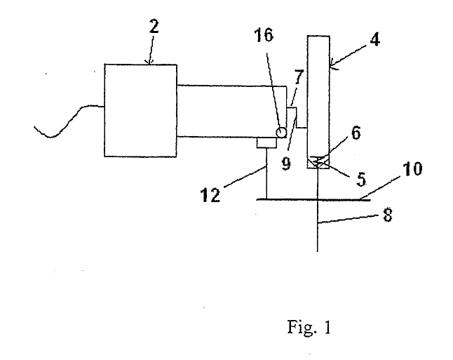

### DETAILED DESCRIPTION OF THE INVENTION

The arrangement according to the present invention comprises a handle 2 and a needle holder member 4 which is adapted to house a hollow needle 8. The needle 8 is preferably provided in the needle holder member 4 by means of a clutching means 5. The clutching means is adapted to readily release the needle 8. For instance, when the needle successfully has penetrated the sampling site and the operator is about to for instance aspirate a sample into the needle (described in further detail below) the operator can, if he so desires, by means of the clutching means release the needle and thus remove the handle and the needle holder member, so that the needle only is present at the sampling site facilitating subsequent operations with the needle. The needle 8 may be an evacuation needle (0.5-3 mm), an aspiration needle (1.2-3.2 mm) or a coarse biopsy needle (3.2-6 mm). The needle 8 may be provided with an obturator (not shown) in order to prevent unwanted material to fill the needle during the insertion of the needle into the bone marrow or the sinuses.

The arrangement comprises longitudinal movement means and rotational movement means (not shown) that are adapted to set the needle 8 in longitudinal movement, preferably sinusoidal, and rotational movement, respectively. The movement means are preferably provided by an electric motor(s) provided in the handle 2. The electric motor can naturally be replaced by any suitable mechanical actuator, powered by for instance pneumatic or electromechanical energy. The longitudinal and rotational movement, respectively, is transferred to the needle 8, preferably via a crank shaft 7 and a connecting rod 9. The length of the crank shaft 7 is of the same length as the amplitude of the longitudinal movement, which is in the order of approximately 0-4 mm. In order to be able to vary the amplitude, said crank shaft can be provided as a readily exchangeable item. The amplitude can also be made as to be automatically variable. This can for instance be made by having a balanced bended crank shaft (not shown in figures) with increasing crank length with respect to the motor. The attachment means (not shown) of the needle holder member (4) to the crank shaft can thus for instance be slideably provided so that the crank length can be readily adjusted if the operator of the arrangement wants to change the amplitude of the longitudinal movement. Naturally said attachment means is provided with means that is adapted to also lock the needle holder member (4) in a certain position when the desired length has been adjusted. Said adjustment can be accomplished also during ongoing longitudinal movement of the needle. The advantages of having a variable amplitude is described in further detail below.

In order to reduce the transmissions of vibrations originating from the needle movements to the handpiece, said arrangement is preferably provided with a damping device (not shown), such as a dampening layer, at (a) preferred site(s) of the arrangement.

The arrangement is also preferably provided with pressure sensing means 6 that in a preferred embodiment is adapted to sense the force experienced by the needle 8 along the longitudinal axis of the needle. The pressure sensing means 6 is preferably in the form of a pressure/force sensor, for example a spring mechanism, that for instance is provided in the needle holder member 4 connected to the needle 8. Another embodiment of the pressure sensing means is described below.

The arrangement is further preferably provided with a stopping means realized by at least one adjustable distance plate 10, arranged perpendicular to the needle 8. The distance plate(s) 10 is/are preferably connected to the handle 2 by means of at least one distance adjusting means 12, preferably a screw, by means of which distance adjusting means 12 the distance between the needle tip and the distance plate(s) is adjustable. The distance plate 10 may be a flat disc of any geometric form provided with a hole, preferably in the centre of the disc, through which hole the needle 8 is provided. The stopping means may also be realized by for instance several distance plates 10 provided as flat discs of any geometric form provided symmetrically around the needle 8. The distance plate(s) 10 has a sufficient extension perpendicular to the needle 8 in order to realize a stable and reliable stop.

The arrangement is further most preferably provided with longitudinal movement stopping means that provides for an active break of the longitudinal movement of the needle 8 by e.g. reducing/stopping the supplied power or short-circuiting the electric motor.

In one embodiment, the arrangement comprises means that are adapted to provide an under pressure to the hollow part of the needle 8. The under pressure source can be an internal under pressure source in order to avoid the use of an external under pressure source that might introduce non sterile particles to the arrangement. The internal under pressure source is adapted to apply under pressure to the hollow part of the needle in order to aspirate a sample in to the needle or facilitate the sampling of a biopsy core. Said means preferably comprises a cavity (not shown), preferably made of plastic and provided as a sterilized consumable article, which is mounted in the needle holder member under spring load such that the cavity wants to expand. When the spring load on the cavity is reduced, under pressure is applied to the hollow part of the needle 8, preferably via a sterilized hose (not shown).

Alternatively the arrangement can be connected to other pre-charged under pressure sources or an external under pressure source adapted to apply under pressure to the hollow part of the needle 8.

In another embodiment, when the arrangement is used for, for instance, evacuation of the sinuses, the arrangement is adapted to be provided with means adapted to apply saline solution to the hollow part of the needle. When the needle successfully has penetrated the predetermined penetration site (described in further detail below), the operator can for instance release the needle 8 by means of the clutching means 5 as described above, whereupon external saline solution means (not shown), for instance a syringe or the like, is applied to the needle. Saline solution is then applied to the hollow part of the needle so that the sinuses are flushed/evacuated by means of the solution. Alternatively, the arrangement can be provided with an internal saline solution means (not shown) adapted to apply saline solution the hollow part of the needle as described above, whereupon the needle need not to be released by means of the clutching means in order for the hollow part of the needle to be provided with saline solution. Any purulent secretion present in the sinuses, can prior to the flushing of said sinuses, if necessary, at least partly be removed for analysis by means of internal or external under pressure sources, such as the ones described above, i.e. said secretion may be removed with the needle released from the needle holding member by using an external under pressure source, as well as be removed with the needle still attached by use of the internal under pressure means. Naturally, when reference is made to saline solution, it is to be understood that any solution used for flushing the sinuses can be used with the inventive arrangement.

In any of the embodiments described above, the handle is also preferably provided with a light source 16, for instance a light-emitting diode or the like, which is adapted to provide a powerful light during the procedure in order to facilitate the procedure for the operator of the arrangement.

The arrangement further comprises control means and a corresponding control panel, preferably provided in the handle 2 and preferably programmable, by means of which the operator of the arrangement is able to control the different means of the arrangement comprising the longitudinal and rotational movement means, the distance adjusting means, the light source etc.

During all procedures with the present invention, it is extremely important that components in contact with the patient are thoroughly sterilized in order to prevent infections. Therefore, the handle 2 is adapted to be fitted into a plastic covering (not shown), provided as a sterilized consumable article. The plastic covering is thus adapted to be tightly fitted around the handle 2. The plastic covering is at the area adjacent the needle 8 provided in such a way that non-sterile particles are prevented from falling into the wound.

Alternatively, the handle might be sterilised via means of autoclavation or contact sterilizing.

Due to the requirements for sterility, means that may introduce non-sterile particles or is in contact with the sample are provided as sterilized consumable articles in a consumable sterilized kit. The consumable kit preferably comprises the needle 8, the pressure sensing means 6, the needle holder member 4, as well as the internal under pressure source comprising the cavity and the hose connected to the needle 8 and the plastic covering, if provided.

During use of the arrangement in any of the procedures described above, the operator of the arrangement directs the needle 8 towards the penetration site. Depending on the penetration site, i.e. if the sample is to be taken from the posterior or anterior iliac crest, sternum or tibia, or if the sinuses are to be evacuated, the needle 8 is held horizontally or vertically towards said site. The operator then may apply a longitudinal movement to the needle 8 by means of the longitudinal movement means, whereupon the reciprocating needle 8 penetrates the skin and subcutaneous tissue and further advances through the bone. Preferably, the longitudinal movement is applied to the needle 8 when the needle is about to penetrate the bone, i.e. no longitudinal movement is applied during the penetration of the needle 8 through skin and subcutaneous tissue. The longitudinal movement frequency is preferably in the range of 30-300 Hz and the inventors of the present invention have in fact found that by for instance applying a longitudinal movement frequency of 250 Hz to the needle, the penetration force needed on the needle 8 for penetrating the bone is reduced by as much as 90%. Thus, the experienced pain of the patient is reduced and the time needed for the needle 8 to penetrate the bone is decreased. Due to the fact that less penetration force needs to be applied to the needle in order to penetrate the bone, coarse needles with a diameter up to 6 mm may be used with the inventive arrangement. The present inventors have shown that the needed penetration force decreases with increasing longitudinal movement frequency and amplitude. That is, with the same longitudinal movement frequency, a higher amplitude will provide for a decreased penetration force needed and vice versa. As the skilled person readily appreciates, it may sometimes be more preferred to, during use of the arrangement, increase/decrease the amplitude of the movement instead of increasing/decreasing the frequency. The amplitude and the frequency of the longitudinal movement can thus be changed independently.

Optionally the operator of the arrangement may apply a rotational movement to the needle 8 by means of the rotational movement means to further enhance the penetration. The longitudinal and rotational movement means may thus be maneuvered independently.

When the needle 8 has penetrated the cortex bone or the first sinus wall, the needle then enters the soft marrow cavity or the sinus cavity respectively. Due to the low penetration force applied to the needle 8, the risk for the needle 8 to also penetrate posterior cortex, i.e. the second bone wall, or the second sinus wall and injure the orbit or underlying blood vessels is significantly reduced.

When the needle 8 enters the soft marrow cavity or the sinus cavity, the resistance of penetration decreases, i.e. the force acting along the longitudinal axis of the needle decreases, whereupon the longitudinal movement stopping means in one embodiment can provide for an active break of the longitudinal movement of the needle 8 by e.g. reducing/stopping the electric power supplied or short circuiting the electric motor. This is accomplished by means of the pressure sensing means 6 that in the preferred embodiment senses the force acting on the needle 8. If the force is below a predetermined level, the pressure sensing means 6 generates a signal to the control means which in turn provides for a reduction/stop of the longitudinal movement of the needle 8.

The distance plate 10 can also be pre-set by means of the distance adjusting means 12 to a predetermined penetration depth. Thus, the distance plate 10 constitutes a hindrance for the needle 8 to penetrate deeper than to the predetermined depth.

Moreover, the arrangement can be provided with means in order for the longitudinal movement stopping means to provide for an active break of the longitudinal movement of the needle 8, when the distance plate 10 is pressed upwards by the force acting on the plate perpendicular to the extension of the distance plate 10, i.e. when the needle 8 has penetrated down to the predetermined depth. This is accomplished by means of having for instance the distance plate 10 provided with pressure sensing means, for instance in the form of a pressure/force sensor, for example a spring mechanism, that will generate a signal to the control means, which in turn provides for an active break of the longitudinal movement of the needle 8 by e.g. reducing/stopping the electric power supplied or short circuiting the electric motor, when the force experienced by the pressure sensing means is over a predetermined level. Naturally, the pressure sensing means adapted to sense the force acting on the plate perpendicular to the extension of the distance plate 10, need not to be provided on the actual distance plate 10, but can be provided at any suitable place of the arrangement as long as said pressure sensing means provides for the functions as described above.

Preferably, if the longitudinal movement of the needle 8 has been actively reduced/stopped, by the longitudinal movement stopping means described above, the longitudinal movement of the needle 8 is prevented to be applied to the needle 8, until, preferably the operator of the arrangement, for instance manually resets the longitudinal movement stopping means and the longitudinal movement may preferably only be applied to the needle 8 as long as a certain force is applied to the needle 8.

If the present invention is used for taking aspirates of the bone marrow, the operator applies an under pressure by means of the internal or external under pressure source to the hollow part of the aspiration needle 8 when the needle is correctly positioned in the bone marrow, whereupon a sample is aspirated into said hollow part. Optionally, the needle may be in the longitudinal and/or rotational movement during aspiration of the sample in order to enhance sample quality and volume. If the aspiration needle is provided with an obturator, said obturator is removed before aspiration of the sample as conventional in the art. The needle is then retracted and the bone marrow aspiration sample may be transferred for examination.

If the present invention is used for taking biopsies of the bone marrow, the needle 8 is a coarse biopsy needle, preferably provided with an obturator. When the needle 8 is correctly positioned at the sampling site, the obturator is removed from the needle. The biopsy core sample enters the hollow part of the needle when it is further inserted to the marrow. Longitudinal movement of the needle, and optionally also the rotational movement, is preferably added to facilitate the entering of the biopsy core into hollow part of the needle 8. A slight under pressure may also by means of the internal or external under pressure source be applied to the hollow part of the needle 8 during the sampling procedure in order to further facilitate the entering of the biopsy core into said hollow part. After the sampling is concluded, the needle is retracted from the sampling site, preferably when in the longitudinal movement and/or the rotational movement and with the slight under pressure applied, in order to effectively prevent the biopsy core to fall out of the needle 8 and thus reducing the risk of leaving the sample in situ.

If the present invention is used for evacuating the sinuses, the operator flushes the cavities with saline water via the hollow part of the evacuation needle 8 when the needle is correctly positioned, as described above. Optionally, the needle may be in the longitudinal and/or rotational movement during the flushing/evacuation of the sinuses in order to improve and speed up the procedure, i.e. to effectively avoid obstruction and decrease resistance in the needle. If the evacuation needle is provided with an obturator, said obturator is removed before flushing as conventional in the art. The needle is subsequently retracted at the end of the procedure, optionally in the longitudinal and/or rotational movement.

It will be understood that the invention is not restricted to the above-described exemplifying embodiments thereof and that several conceivable modifications of the invention are possible within the scope of the following claims.

1. An arrangement for taking a sample of bone marrow and/or evacuating the sinuses, comprising a handle (2) with a needle holder member (4) provided with a hollow needle (8) adapted to penetrate the bone characterized in that the arrangement comprises longitudinal movement means adapted to apply a longitudinal movement to the needle (8), preferably when the needle is penetrating the bone.

2. An arrangement according to claim 1 characterized in that the longitudinal movement frequency is in the range of 30-300 Hz.

3. An arrangement according to claim 1, characterized in that the amplitude of the longitudinal movement is in the range of 0-4 mm and in that said amplitude is variable during longitudinal movement of the needle (8).

4. An arrangement according to claim 1 characterized in that the arrangement comprises rotational movement means adapted to apply a rotational movement to the needle (8).

5. An arrangement according to claim 1 characterized in that the longitudinal movement and/or the rotational movement is applied to the needle (8):
during the sampling of bone marrow and/or during evacuation of the sinuses, and/or
during the retraction of the needle (8) from the penetration site.

6. An arrangement according to claim 1 characterized in that the arrangement is provided with stopping means that prevents the needle (8) to penetrate deeper into the bone than to a predetermined depth, said stopping means comprises at least one adjustable distance plate (10) arranged perpendicular to the needle (8) with a certain distance between the distance plate(s) (10) and the needle tip, whereupon the stopping means further comprises distance adjusting means (12) to adjust the distance between the distance plate(s) and the needle tip.

7. An arrangement according to claim 1 characterized in that the arrangement is provided with means adapted to generate a signal to a control means when the needle (8) has penetrated down to a predetermined depth and that said control means then provides for a reduction and/or stop of the longitudinal movement of the needle.

8. An arrangement according to claim 7 characterized in that the means adapted to generate the signal to the control means when the needle has penetrated down to the predetermined depth, is in the form of pressure sensing means (6) connected to the needle (8), and arranged to sense a force experienced by the needle along the longitudinal axis of the needle, whereupon said pressure sensing means generates said signal to the control means when said force is below a predetermined level.

9. An arrangement according to claim 6 characterized in that the means adapted to generate the signal to the control means when the needle has penetrated down to the predetermined depth, is in the form of a pressure sensing means arranged to sense the force experienced by the distance plate(s) (10) when the needle has penetrated down to the predetermined depth, and when the force is over a predetermined level generates the signal to the control means.

10. An arrangement according to claim 8 characterized in that the pressure sensing means is a spring mechanism.

11. An arrangement according to claim 1 characterized in that the arrangement comprises an internal under pressure source adapted to apply an under pressure to the hollow part of the needle (8).

12. An arrangement according to claim 2, characterized in that the amplitude of the longitudinal movement is in the range of 0-4 mm and in that said amplitude is variable during longitudinal movement of the needle (8).

13. An arrangement according to claim 2, characterized in that the arrangement comprises rotational movement means adapted to apply a rotational movement to the needle (8).

14. An arrangement according to claim 3, characterized in that the arrangement comprises rotational movement means adapted to apply a rotational movement to the needle (8).

15. An arrangement according to claim 2 characterized in that the longitudinal movement and/or the rotational movement is applied to the needle (8):
during the sampling of bone marrow and/or during evacuation of the sinuses, and/or
during the retraction of the needle (8) from the penetration site.

16. An arrangement according to claim 3 characterized in that the longitudinal movement and/or the rotational movement is applied to the needle (8):
during the sampling of bone marrow and/or during evacuation of the sinuses, and/or
during the retraction of the needle (8) from the penetration site.

17. An arrangement according to claim 4 characterized in that the longitudinal movement and/or the rotational movement is applied to the needle (8):
during the sampling of bone marrow and/or during evacuation of the sinuses, and/or
during the retraction of the needle (8) from the penetration site.

18. An arrangement according to claim 2 characterized in that the arrangement is provided with stopping means that prevents the needle (8) to penetrate deeper into the bone than to a predetermined depth, said stopping means comprises at least one adjustable distance plate (10) arranged perpendicular to the needle (8) with a certain distance between the distance plate(s) (10) and the needle tip, whereupon the stopping means further comprises distance adjusting means (12) to adjust the distance between the distance plate(s) and the needle tip.

19. An arrangement according to claim 2 characterized in that the arrangement is provided with means adapted to generate a signal to a control means when the needle (8) has penetrated down to a predetermined depth and that said control means then provides for a reduction and/or stop of the longitudinal movement of the needle.

20. An arrangement according to claim 9 characterized in that the pressure sensing means is a spring mechanism.
